(19) United States
(12) Patent Application Publication
(10) Pub. No.: US 2004/0219109 A1
(43) Pub. Date: Nov. 4, 2004

(54) Printing identification of incorporated medications onto medicated chewing gums, medicated candies, and other medicated edible products

(76) Inventor: Edwin Burton Hatch, West Bend, WI (US)

Correspondence Address: Mr. Edwin Burton Hatch, 1610 Tumbleweed Circle, West Bend, WI, 53095, US

(21) Appl. No.: 10/789,015

(22) Filed: Feb. 28, 2004

Related U.S. Application Data
(60) Provisional application No. 60/452,816, filed on Mar. 10, 2003.

(51) Int. Cl. A61K 9/68; A61K 9/42; A61J 1/00

(52) U.S. Cl. 424/48; 427/2.23

(57) ABSTRACT

Edible or inert or nontoxic inks or other materials are printed or otherwise transferred onto the surfaces of medicated chewing gums, medicated candies, and other medicated edible products so as to provide easy and immediate visual identification of the medicaments and the dosages of the medicaments present and to identify the manufacturer of the medicated product and provide such data as may be needed to trace the batch of manufacture. Medicaments may be incorporated into the ink or material transferred onto chewing gums, candies, and other non-medicated edible products so as to create medicated chewing gums, medicated candies, and other medicated edible products. This printed identification may be done in various colors to further aid in the visual identification of the medicaments present and further guarantee safe medication of the patient.

This is a continuation of U.S. Provisional Patent Application No.: 60/452.816; Filing Date: Mar. 10, 2003; Name of Applicant: Edwin Burton Hatch, West Bend, Wis.; Title of Invention: “Medicated chewing gum identification and method of production”. Copy of Provisional Patent Documents are attached.

This invention concerns the identification of medicament present in medicated chewing gums, medicated candies, and other medicated food products used to medicate a patient. In this invention edible or inert or nontoxic inks or other materials are printed or otherwise transferred onto the surfaces of medicated chewing gums, medicated candies, and other medicated edible products so as to provide easy and immediate visual identification of the medicaments and dosages present and to identify the manufacturer of the medicated product and provide such data as may be needed to trace the batch of manufacture. This printing may be done in various colors to further aid in the visual identification of the medicament present and further guarantee safe medication of the patient. This invention also teaches that a medicament may be incorporated into the ink or material transferred onto the surfaces of a non-medicated edible product so as to there by create a medicated product.

### REFERENCES CITED

1. “Methods and products for delivering a medicament to an individual are provided.” Here medicaments [drugs] are in the coating surrounding the gum center but no visual external identification of the incorporated drugs is provided:

| | | |
|---|---|---|
| 6,465,003 | Oct., 15, 2002 | Ream et al. |

2. “A method for producing a chewing gum with a controlled release of caffeine, as well as the chewing gum so produced, is obtained by physically modifying caffeine's properties by coating and drying.” Here Caffeine particles are incorporated into chewing gum but no visual external identification of the incorporated drug is provided:

| | | |
|---|---|---|
| 6,444,241 | Sep., 3, 2002 | Tyrpin |

3. “The invention relates to stable, pharmaceutically usable chewing gum formulations which contain acetylsalicylic acid [ASA] as the active compound, and to a process for their preparation.” Here acetylsalicylic acid [ASA] is incorporated into chewing gum but no visual external identification of the incorporated drug is provided:

| | | |
|---|---|---|
| 5,922,347 | Jul., 13, 1999 | Hausler |

4. “The dental products of this invention can be used to treat and prevent periodontal disease.” Here mixtures of poleaxes, poloaxemer congerers and xylitol are incorporated into chewing gum but no visual external identification of the drugs is provided:

| | | |
|---|---|---|
| 5,900,230 | May, 4, 1999 | Cutler |

5. “The present invention relates to a medicated chewing gum comprising pharmaceutically active agent incorporated therein.” Here a process is described wherein drugs are incorporated into chewing gum but no visual external identification of the incorporated drugs is provided:

| | | |
|---|---|---|
| 5,846,557 | Dec., 8, 1998 | Eisenstad |

6. “A solid or semi-solid bioadherent, orally ingestible drug delivery system containing a water-in-oil system having at least two phases, one phase comprises from about 25% to about 75% by volume of an internal hydrophilic phase and the other phase comprises from about 25% to about 75% by volume of an external hydrophilic phase and wherein the external hydrophobic phase is comprised of three components, a) an emulsifier, b) a glyceride ester and c) a wax material.” Here a process is described wherein drugs are incorporated into chewing gum but no visual external identification of the incorporated drugs is provided:

| | | |
|---|---|---|
| 5,554,380 | Sep., 10, 1996 | Cuca et al. |

7. “A chewing gum that contains porous polymeric beads impregnated with active gum ingredients together with a method of making such a chewing gum.” Here a process is described wherein pharmaceutical agents are incorporated into chewing gum but no visual external identification of the incorporated pharmaceutical agents is provided:

| | | |
|---|---|---|
| 5,154,927 | Oct., 13, 1992 | Song |

8. “A Medicament adsorb ate and process for making same.” Here a process is described wherein pharmaceutical agents are incorporated into a lozenge, a tablet, toffee, nougat, chewing candy, and chewing gum but no visual external identification of the incorporated pharmaceutical agents is provided:

| | | |
|---|---|---|
| 4,753,800 | Jun., 28, 1988 | Mozda |

9. “A chewing gum composition adapted to supply a medicament to the oral cavity for local application thereto or for buccal adsorption of said medicament which comprises a chewing gum base, an orally administrable medicament, a taste masking generator of carbon dioxide and optionally a taste bud desensitizing composition.” Here a process is described wherein medicaments are incorporated into chewing gum but no visual external identification of the incorporated drugs is provided:

| | | |
|---|---|---|
| 4,639,368 | Jan., 27, 1987 | Niazi |

### BACKGROUND OF INVENTION

Medicated chewing gums, medicated candies, and other medicated edible products have been shown to be an effective means of delivering medicaments [drugs] into the blood primarily through the mucous membranes of the mouth and secondarily through the gastrointestinal track. These medicated edible products have significant advantages for patients who have difficulty swallowing tablets or capsules. However, serious new safety issues arise as doctors prescribe medicated chewing gums, medicated candies, and other medicated edible products for elderly patients, for mentally impaired, or for visually impaired patients who may be easily confused when taking medications and thereby miss there medications or ingest multiple dosages. It is also feared that children may eat these medicated chewing gums, medicated candies, and other medicated edible products mistaking them for similar common non-medicated products. This is especially true of medicated chewing gums and medicated candies through which children may ingest dangerous amounts of medicaments. Better means of visual identification of medicaments and dosages of those medicaments in these medicated edible products are clearly needed in order to resolve these new consumer safety issues. It is also desirable and necessary to be able to trace the manufacturing sources and batch numbers of medicated chewing gums, medicated candies, and other medicated edible products in order to be able to recall bad batches of these medicated products as well as to establish legal responsibility in various situations.

### SUMMARY OF INVENTION

1. In order to resolve the above-described safety issues, in this invention here in taught edible, inert, or nontoxic inks or other materials are printed onto the surfaces of medicated chewing gums, medicated candies, and other medicated edible products by various printing processes in various unique colors and/or patterns so as to provide clear visual identification of the medicaments [drugs] and dosages of those medicaments contained therein for the protection of patient, for the information of and confirmation by medical personnel, and to differentiate these medicated edible products from similar non-medicated edible products for improved adult and child safety.

2. It is also herein taught that the above mentioned printing may include bar codes, or other marking systems, for the purpose of identifying the source of manufacture, the production batch number, and such other data as may be desirable or required in order to trace the manufacture of those medicated chewing gums, medicated candies, and/or other medicated edible products.

3. It is also herein taught that the above mentioned edible, inert, or nontoxic inks or other materials may be heated and/or melted prior to being printed onto the surfaces of medicated chewing gums, medicated candies, and other medicated edible products and that these heated materials cool and solidify upon being printed or transferred onto the above mentioned medicated chewing gums, medicated candies, and other medicated edible products for the purpose of speeding up and simplifying the manufacturing processes.

4. It is also here in taught that medicaments may be incorporation into the above mentioned edible, inert, or nontoxic inks or other materials which are subsequently printed or transferred onto the surfaces of un-medicated chewing gums, un-medicated candies, and other un-medicated edible products, by various printing processes in various unique colors and/or patterns to provide clear visual identification of the medicaments [drugs] and dosages contained therein to patient and medical personnel, thereby creating or producing medicated chewing gums, medicated candies, and/or other medicated edible products.

### DETAILED DESCRIPTION

The accompanying drawings further clarify the embodiment of the present invention:

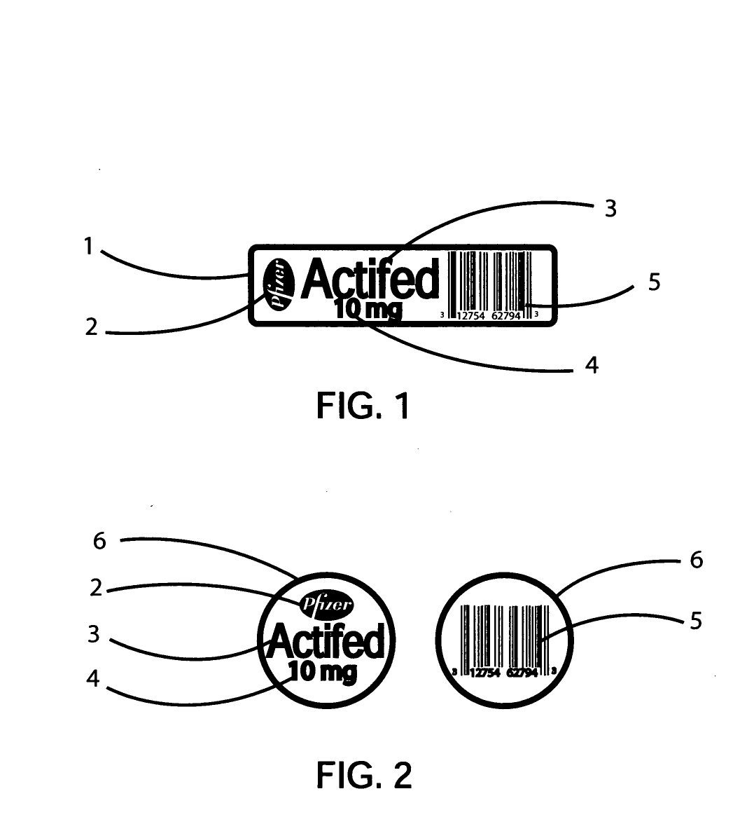

FIG. 1 shows a typical piece of medicated chewing gum [1] with the manufacturer's logo [2], the name of the medication [3], the dosage of the medication [4], and the barcode of the batch of manufacture [5] printed onto the surface of the said typical piece of medicated chewing gum .

FIG. 2 shows a typical piece of medicated candy [6] with the manufacturer's logo [2], the name of the medication [3], and the dosage of the medication [4], printed onto the front surface there of, and with the barcode of the batch of manufacture [5] printed onto the back surface[6] of the said typical piece of medicated candy.

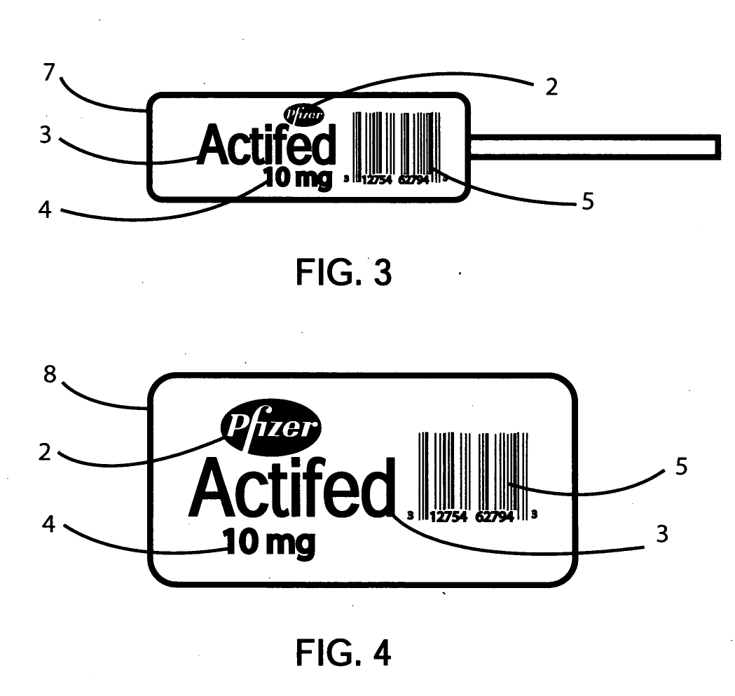

FIG. 3 shows a typical medicated candy lollypop [7] with the manufacturer's logo [2], the name of the medication [3], the dosage of the medication [4], and the barcode of the batch of manufacture [5] printed onto the surface of the said typical medicated candy lollypop.

FIG. 4 shows a typical medicated cracker or other medicated edible product [8] with the manufacturer's logo [2], the name of the medication [3], the dosage of the medication [4], and the barcode of the batch of manufacture [5] printed onto the surface of the said typical medicated cracker or other medicated edible Product.

Because many variations, modifications, and different embodiments may be made within the scope of the invention herein taught and detailed in accordance with the descriptive requirements of the law, it is to be understood that this said embodiment here in detailed is to be interpreted as being illustrative and not limiting.

We claim:

1. The printing of edible, inert, or non-toxic inks or materials onto the surfaces of medicated chewing gums, medicated candies, and/or other medicated edible products by, but not limited to, silk screen printing processes, ink jet printing processes, offset printing processes, and/or other printing or transfer processes in various unique colors and/or patterns for the purpose of visually identifying the medicaments and dosages of said medicaments contained in the said medicated chewing gums, medicated candies, and other medicated edible products and which said printing may include bar codes, or other marking systems, for the purpose of identifying the source of manufacture, the production batch number, and such other data as may be necessary to trace manufacture of the said medicated chewing gums, medicated candies, and other medicated edible products.

2. The printing of edible, inert, or non-toxic inks or materials of claim 1 wherein the said materials are printed onto the surfaces of said medicated chewing gums, medicated candies, and other medicated edible products by means of a rotary silk screen printing process.

3. The printing of edible, inert, or non-toxic inks or materials which are heated prior to being printed or transferred onto the surfaces of medicated chewing gums, medicated candies, and/or other medicated edible products by, but not limited to, silk screen printing processes, ink jet printing processes, offset printing processes, and/or other printing or transfer processes in various unique colors and/or patterns for the purpose of visually identifying the medicaments and dosages of said medicaments contained in the said chewing gums, medicated candies, and other medicated edible products and which said printing may include bar codes, or other marking systems, for the purpose of identifying the source of manufacture, the production batch number, and such other data as may be necessary to trace the manufacture of the said medicated chewing gums, medicated candies, and other medicated edible products, and which said printed heated materials cool and solidify upon being printed or transferred onto the said medicated chewing gums, medicated candies, and other medicated edible products there by speeding up and simplifying the manufacturing processes.

4. The incorporation of medicaments into edible, inert, or nontoxic inks or other materials which are subsequently printed or transferred onto the surfaces of un-medicated chewing gums, un-medicated candies, and/or other un-medicated edible products in various unique colors and/or patterns by, but not limited to, silk screen printing processes, ink jet printing processes, offset printing processes, or other printing or transfer processes so as to produce or create medicated chewing gums, medicated candies, and other medicated edible products, which said printed colors and/or patterns provide visual Identification of the medicaments and dosages of said medicaments contained in the said medicated chewing gums, medicated candies, and other medicated edible products and which said printing may include bar codes, or other marking systems, for the purpose of identifying the source of manufacture, the production batch number, and such other data as may be necessary to trace the manufacture of the said medicated chewing gums, medicated candies, and other medicated edible products.

5. The printing of medicaments incorporated into the edible, inert, or nontoxic inks of claim 4 which are heated prior to being printed or transferred onto the surfaces of the said un-medicated chewing gums, un-medicated candies, and/or other un-medicated edible products where in the said heated medicated materials cool and solidify after said printing so as to produce or create the said medicated chewing gums, medicated candies, and other medicated edible products.
